(19) United States
(12) Patent Application Publication
(10) Pub. No.: US 2026/0053878 A1
(43) Pub. Date: Feb. 26, 2026

(54) PROCESS FOR EXTRACTION OF ACTIVE COMPOUNDS FROM PLANT BIOMASS

(71) Applicant: Neural Therapeutics Inc., Toronto (CA)

(72) Inventors: Anthony DeMeo, Las Vegas, NV (US); Kevin Roblek, Las Vegas, NV (US)

(21) Appl. No.: 19/105,331

(22) PCT Filed: Aug. 28, 2023
(86) PCT No.: PCT/CA2023/051137

§ 371 Date: Feb. 20, 2025

Related U.S. Application Data
(60) Provisional application No. 63/401,352, filed on Aug. 26, 2022.

(51) Int. Cl. A61K 36/33; A61K 31/137; C07C 213/10

(52) U.S. Cl. CPC A61K 36/33; A61K 31/137; C07C 213/10; A61K 2236/13; A61K 2236/15; A61K 2236/17; A61K 2236/33; A61K 2236/39; A61K 2236/53; A61K 2236/55

(57) ABSTRACT

The present disclosure is generally directed to the extraction of active compounds from plant biomass. In particular, the present disclosure is directed to the extraction of active compounds from plant biomass using solvents of different polarity.

### CROSS-REFERENCE TO RELATED APPLICATION

This application claims the benefit of priority to U.S. Provisional Application No. 63/401,352, filed Aug. 26, 2022, the contents of which is incorporated herein by reference in its entirety.

### FIELD

The present disclosure is generally directed to the extraction of active compounds from plant biomass. In particular, the present disclosure is directed to the extraction of active compounds from plant biomass using solvents of different polarity.

### INTRODUCTION

Plant extracts are traditionally made by exposing the plant material to carbon dioxide, butane, propane or other solvents in a vessel or mixer to leach the extracts from the plants. Other processes, such as agitation, have been developed in an attempt to liberate the compounds from the plants. These and other traditional techniques often suffer from issues such as low yield, safety issues and/or contaminants that adversely affect the flavor or efficacy of the resulting extracts.

There remains a need for a method of extracting active compounds, such as mescaline, tyramine, N-methyltyramine, hordenine, N-methylmescaline, anhalinine, O-methylanhalinine, anhalidine, anhalamine, anhaloridine, pellotine, anhalonine, lophophorine, other alkaloids, cannabinoids, and other plant and fungi compounds, that addresses the problems of the prior art, and in particular, resolves the issues of low yield and low purity.

### SUMMARY

The present disclosure is directed to a process for the extraction of active compounds from a plant and/or and fungi material, resulting in increased yields and purity of the extracted compounds. In one embodiment of the disclosure, there is included a process for the extraction of active compounds from plant material, the process comprising:
a) separating the epidermis from the plant material;
b) isolating (separating) the chlorenchyma of the plant;
c) drying, optionally by freeze drying, the chlorenchyma to a moisture level of less than 10%;
d) reducing the particle size of the dried chlorenchyma to obtain a powder;
e) mixing the powder with a non-polar solvent to form a mixture having a solid phase and a liquid phase, wherein the solid phase comprises the active compounds and the liquid phase comprises hydrophobic compounds;
f) agitating, optionally by sonicating, the mixture at low power to reduce the particle size of the solid phase;
g) separating the solid phase from the liquid phase, optionally by centrifugation;
h) extracting the active compounds from the solid phase by mixing the solid phase with an alcohol, such as methanol, to form an alcoholic mixture;
i) separating, optionally by centrifuging, the alcoholic mixture to obtain a first alcoholic extract and a depleted solid phase;
j) separating, optionally by filtering, the alcoholic extract to obtain a filtered alcoholic extract;
k) optionally repeating steps g)-i) on the depleted solid phase to obtain a second filtered alcoholic extract;
l) combining the first and second alcoholic extracts and removing, optionally by evaporating, the alcohol to obtain a crude oil (extract) comprising the active compounds;
m) mixing the crude oil (extract) with ethyl acetate and acidulated water to form a crude mixture;
n) agitating, optionally by sonication (either with an ultrasonic probe or in an ultrasonic bath), the crude mixture at low power to obtain a water phase and a first ethyl acetate phase;
o) separating the water phase from the ethyl acetate phase;
p) mixing the water phase with a base to obtain a basified water phase having a pH greater than 12.0;
q) mixing the basified water phase with ethyl acetate and separating therefrom a second ethyl acetate phase; and
r) combining the first and second ethyl acetate phases and removing the ethyl acetate to obtain a purified crude oil comprising the active compounds.

In one embodiment, the plant material is a psilocybin-containing fungi plant material.

In one embodiment, in step (b) the parenchymal tissue is isolated from the plant material. A person skilled in the art would understand that the chlorenchyma is synonymous with the chlorophyllaceous cortical parenchyma.

In one embodiment, the plant material is a cannabis plant or an Echinopsis plant or Trichoceurus or Lophophora plant, Pereskia such as Pereskia aculeata, Acacia such as Acacia berlandieri.

In another embodiment, the plant material is Echinopsis and the purified crude oil contains at least about 90% of the mescaline in the Echinopsis plant.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 is a flow chart detailing an extraction process of the disclosure; and

FIG. 2 shows a chromatogram of mescaline isolated using the procedure in Example 2 on a Shimadzu LC-2030 NT (top chart) and a standard of mescaline (bottom chart).

### DESCRIPTION OF VARIOUS EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “non-polar solvent” as used herein refers to any compound, or compound mixture, that is generally not miscible with water. Such non-polar solvent compounds include, but are not limited to, petroleum ether, ethyl acetate, dichloromethane, toluene, hexane, pentane, and/or dimethyl ether.

The term “active compound” as used herein refers to compounds present within the plant material which could be useful in the preventive or curative treatment of diseases, conditions and/or disorders, and could be used as pharmaceuticals or nutraceuticals, or otherwise has positive affects on a subject's health.

The term “plant material” as used herein refers to material derived from plants, and includes all parts of the plants including, roots, stalks, leaves, vegetables, fruits, seeds etc.

The term “hydrophobic compounds” as used herein refers to compounds which are soluble in non-polar solvents, and includes, for example, waxes, fats and/or terpenes.

The term “alcohol” as used herein generally refers to lower chain alcohols and includes methanol, ethanol, propanol, iso-propanol, butanol, etc.

The term “acidified water” as used herein refers to water having an acidic pH, for example, below 7 pH.

The term “basified water” as used herein refers to water having a basic pH, for example, above 7 pH.

### Extraction Processes

The present disclosure is directed to process for extracting active compounds from plant material. In one embodiment, the process is directed to extracting active compounds from the parts of a plant in which the active compounds are most concentrated. For example, in the Echinopsis plant genus (cactuses), also referred to as Trichocereus genus, the active compound mescaline and other alkaloids are concentrated in the chlorenchyma of the cactus plant matter. Separating these parts of the plant matter before extracting the active compounds results in higher yields and purity of the active compounds. In one embodiment, the plant material is a plant material containing alkaloids.

In one embodiment, the plant material is from a globular cacti such as Lophophora williamsii (peyote). In another embodiment, the plant material is from arborescent and columnar cacti, such as Echinopsis and all species of Echinopsis including, Echinopsis lageniformis, Echinopsis macrogona, Echinopsis pasacana, Echinopsis pachanoi, Echinopsis peruviana or Echinopsis scopulicola. In another embodiment, the plant material is any cactus containing mescaline.

In another embodiment, the plant material is a psilocybin-containing fungi material.

In another embodiment, the plant material is a cannabis plant material containing cannabinoids such as tetrahydrocannabinol (THC), cannabinol (CBN), cannabidiol (CBD), cannabigerol (CBG), and cannabichromene (CBC), tetrahydrocannabivarin (THCV), cannabivarin (CBV), cannabidivarin (CBDV), as well as other terpenes.

In another embodiment, the process of the present disclosure improves the extraction efficiency, or total yield, and chemical purity of the final extract of botanical extractions of plant material, such as for example Echinopsis plant material. In one embodiment, multiple solvents having a gradient scale are utilized beginning with solvents or solutions low on the gradient scale (non-polar) and ending with a solution that has a polarity index of at least 9.0.

In another embodiment, the process of the present disclosure involves using different solvents having different polarities (a polarity gradient) and specifically, beginning with less polar or non-polar solvents (such as petroleum ether, ethyl acetate or toluene) to more polar solvents, including alcohols (such as methanol) and protic solvents and acid/base solvents (such as diluted hydrochloric acid or sodium hydroxide solutions). In one embodiment, the polarity gradient aids in removing undesirable compounds in the plant material early in the process, and therefore prevents downstream products from becoming too difficult and complex to work.

In another embodiment, the process increases solvent penetration of the plant material by reducing the size of the biomass, opening the cellular walls of the biomass and utilizing methods of agitation (for example, ultrasonic and centrifugal). In another embodiment, a defatting step is introduced early in the process to increase solubilization of the desired molecules, and to prevent potential contamination of the botanic extract and isolate.

In another embodiment, the plant material, such as a cactus, is prepared to ensure that alkaloids and other desirable compounds are available to be solubilized during the extraction steps. In one embodiment, by preventing the interior flesh and cuticle from being present during the further extraction steps, this avoids compounds in the final extract which may lead to nausea/purging commonly associated with mescaline consumption. In a further embodiment, the biomass is then powderized which allows the solvent to have higher penetration.

In another embodiment, the process includes a defatting step using a non-polar solvent, such as petroleum ether, which has low affinity for the desired alkaloids in the plant material. In one embodiment, by removing waxes, fats, terpenes and oils, this ensures that further extraction solutions are reduced, or free of, mucilage that can cause cacti extracts to be difficult to work with.

In another embodiment of the disclosure, the process includes a step in which the plant material is agitated to open the cells walls of the plant material. In one embodiment, the plant material is sonicated a low to medium power, for example between 50 W and 100 W, for example, at 90 W. In one embodiment, using sonication at lower powers reduces the particle size of the plant material and opens up the cell walls of the plant materials and allows for the solvents to solubilize the active compounds or hydrophobic compounds (depending on the step). Sonicating at lower powers also avoids the generation of heat and avoids the potential for labile ester bonds in the active compounds to break. In another embodiment, ultrasonic waves produce strong cavitation effects, mechanical vibration, perturbation effects, high acceleration, emulsification, diffusion, and stirring effects, as a result of the motion frequency and speed of molecules, thereby strengthening solvent penetration.

In another embodiment of the disclosure, the plant material is mixed with a non-polar solvent early in the process as a defatting step to remove hydrophobic compounds. Conducting the defatting step early in the process avoids problems compared to when a defatting step is conducted later in an extraction process; in particular, defatting later in an extraction process results in a complex mixture of plant compounds, proteins, polysaccharides and other cell components, making it difficult to extract the active compounds, thereby lowering yields.

In another embodiment, once the extract is defatted, a full spectrum extraction is conducted to extract a wide range of compounds including 3,4-dimethoxyphenethylamine, 3-methoxytyramine, 4-hydroxy-3-methoxyphenethylamine, 4-hydroxy-3,5-dimethoxyphenethylamine, anhalonidine, anhalinine, hordenine and tyramine. In addition, small amounts of mescaline will also be in the initial solution due to the moderate solubility of mescaline in alcohols. In one embodiment, this step increases the overall extraction yield of mescaline.

In another embodiment, an acidic water and ethyl acetate wash allows for the separation of the desirable compounds from the undesirable compounds from the methanolic botanical extract, which allows for a full spectrum botanical extract, which can further be subjected to chromatography to isolate crystalline mescaline citrate.

In one embodiment, the biomass will be free of undesirable compounds due to their solubility in non-polar solvents, which leads to an increase in the solubility of mescaline salts during acid/base extraction. In one embodiment, the acid/base extraction focuses on extracting only mescaline salts and a secondary purification step is required for maximizing the chemical purity of the isolated mescaline which results in mescaline fumarate.

Accordingly, by using the advantageous steps described above, the present disclosure is directed to a process which increases the yields and purity of the desirable active compounds. Therefore, in one embodiment of the disclosure, there is included a process for the extraction of active compounds from plant material, the process comprising:
a) separating or isolating the chlorenchyma of the plant material;
b) drying, optionally by freeze drying, the chlorenchyma to a moisture level of less than 10%;
c) reducing the particle size of the dried chlorenchyma to obtain a powder;
d) mixing the powder with a non-polar solvent to form a mixture having a solid phase and a liquid phase, wherein the solid phase comprises the active compounds and the liquid phase comprises hydrophobic compounds;
e) agitating, optionally by sonication, the mixture to reduce the particle size of the solid phase;
f) separating the solid phase from the liquid phase;
g) extracting the active compounds from the solid phase by mixing the solid phase with an alcohol, such as methanol or ethanol, to form an alcoholic mixture;
h) separating, optionally by centrifuging, the alcoholic mixture to obtain a first alcoholic extract and a depleted solid phase;
i) optionally separating, optionally by filtering, the alcoholic extract to obtain a filtered alcoholic extract;
j) optionally repeating steps g)-i) on the depleted solid phase to obtain a second filtered alcoholic extract;
k) combining the first and second alcoholic extracts and removing, optionally by evaporating, the alcohol to obtain a crude oil comprising the active compounds;
l) mixing the crude oil with ethyl acetate and acidified water to form a crude mixture;
m) agitating, optionally by sonication, the crude mixture to obtain a water phase and a first ethyl acetate phase;
n) separating the water phase from the ethyl acetate phase;
o) mixing the water phase with a base to obtain a basified water phase having a basic pH, optionally greater than 12.0;
p) mixing the basified water phase with ethyl acetate and separating therefrom a second ethyl acetate phase; and
q) combining the first and second ethyl acetate phases and removing the ethyl acetate to obtain a purified crude oil comprising the active compounds.

In another embodiment, the epidermis is removed from the plant material before beginning the process.

In one embodiment, the chlorenchyma is isolated from the innermost portions of the plant material.

In one embodiment, in step (b), the chlorenchyma is dried in an oven, optionally at low temperature, or freeze dried to a moisture level of less than about 15%, or less than about 10%, or less than about 5%, or about 0-5%, or about 0-10% by weight.

In one embodiment, in step (c), the particle size of the dried chlorenchyma is reduced to obtain a powder having a particle size of less than 30 μm, or less than 50 μm, or less than 100 μm. In one embodiment, the particle size is reduced by hand such as by using a scalpel to reduce the particle size (and avoid contamination), or by pulverizing, milling or grinding.

In one embodiment, in step (e), the mixture is agitated to further reduce the particle size of the solid phase. In one embodiment, the mixture is agitated by shaking or by using sound waves such as by sonicating the mixture at low power to reduce the particle size of the solid phase.

In one embodiment, in step (f), the solid phase is separated from the liquid phase by filtering or by centrifuging.

In one embodiment, in step (h), the alcoholic mixture is separated to obtain a first alcoholic extract by centrifuging or by filtering.

In one embodiment, in step (k), the alcohol in the alcoholic extracts are removed by evaporating the alcohol to obtain the crude oil.

In one embodiment, in step (m), the crude mixture is agitated to further release active compounds. In one embodiment, the crude mixture is agitated by stirring, shaking or by sound waves such as sonication at low power to obtain a water phase and a first ethyl acetate phase.

In one embodiment, the purified crude oil is further mixed with an acid to crystallize the active compounds.

In one embodiment, the crystallized active compounds are filtered.

In one embodiment, the plant material is a cannabis plant or an Echinopsis plant.

In one embodiment, the active compounds are cannabinoids.

In one embodiment, the active compounds are mescaline and/or other alkaloids.

In one embodiment, the chlorenchyma is freeze dried to a moisture level of between 0-10.0%, or between 0-5.0%.

In one embodiment, the chlorenchyma are reduced in particle size by milling, pulverizing or grinding.

In one embodiment, the mixture in step (e) is sonicated at a power of 50 watts to 100 watts.

In one embodiment, the mixture is sonicated at a power of 90 watts.

In one embodiment, the solid phase is separated from the liquid phase in step (f) by centrifugation.

In one embodiment, the alcohol in step (g) is methanol.

In one embodiment, the alcoholic extract in step (i) is filtered through diatomaceous earth.

In one embodiment, the crude mixture in step (m) is sonicated at a power of 50 watts to 100 watts. In one embodiment, the crude mixture is sonicated at a power of 90 watts.

In one embodiment, the non-polar solvent is petroleum ether, dichloromethane, pentane, hexane or mixtures thereof.

In one embodiment, the non-polar solvent is petroleum ether.

In another embodiment, the plant material is Echinopsis and the purified crude oil contains at least about 90% of the mescaline in the Echinopsis plant.

In another embodiment, the present disclosure is directed to compositions containing specific amounts of mescaline and other alkaloids.

In another embodiment, the mescaline can be isolated as mescaline citrate, mescaline hydrochloride or mescaline fumurate. In one embodiment, the isolated mescaline from the procedure is mixed with citric acid and isolated to obtain mescaline citrate. In another embodiment, the mescaline from the procedure is mixed with hydrochloric acid and isolated to form mescaline hydrochloride. In another embodiment, mescaline fumurate is obtained from mescaline hydrochloride by contacting mescaline hydrochloride with fumaric acid to isolate and obtain mescaline fumurate.

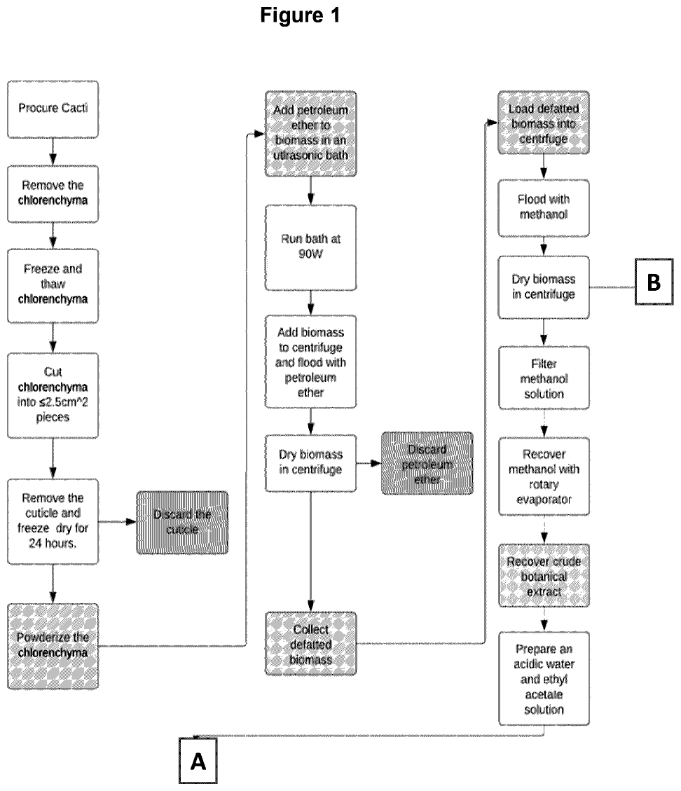

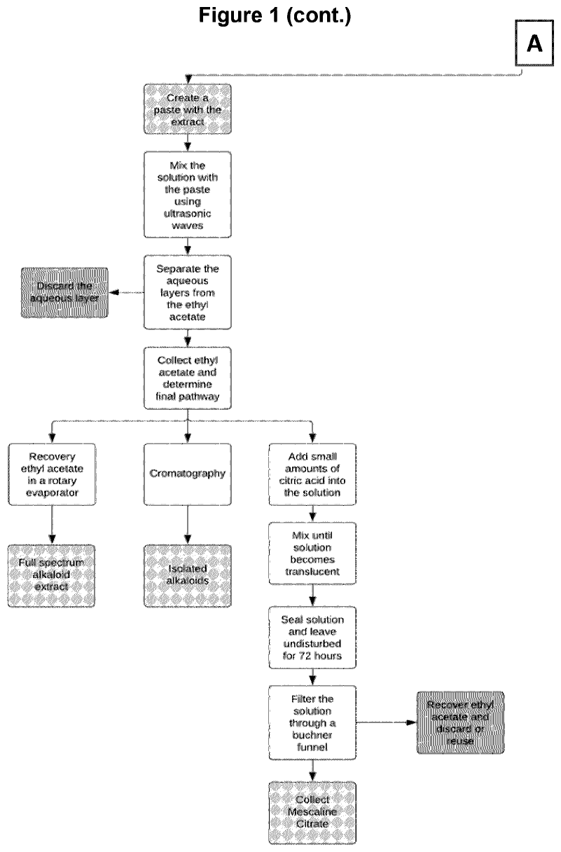

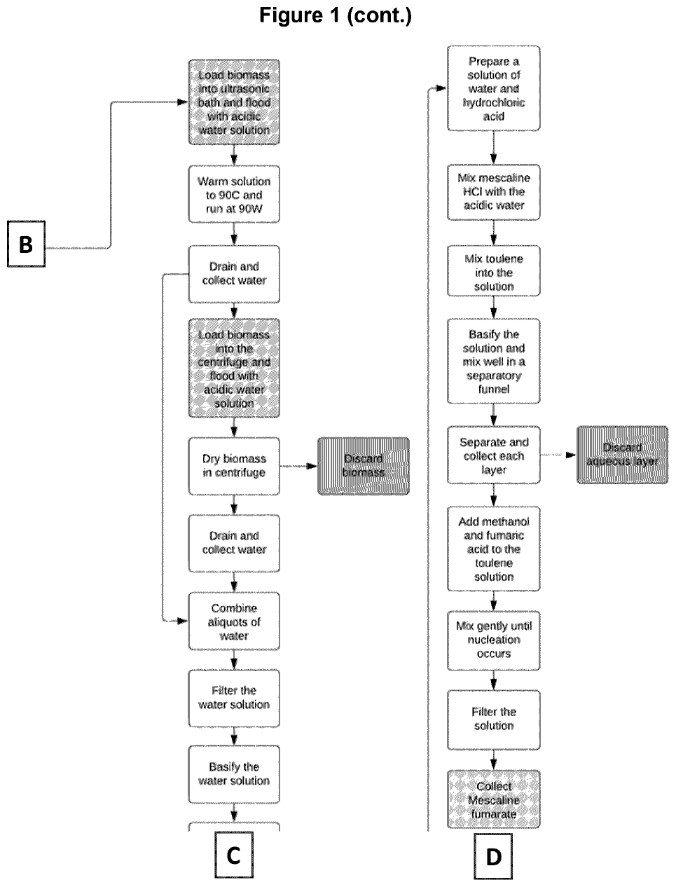

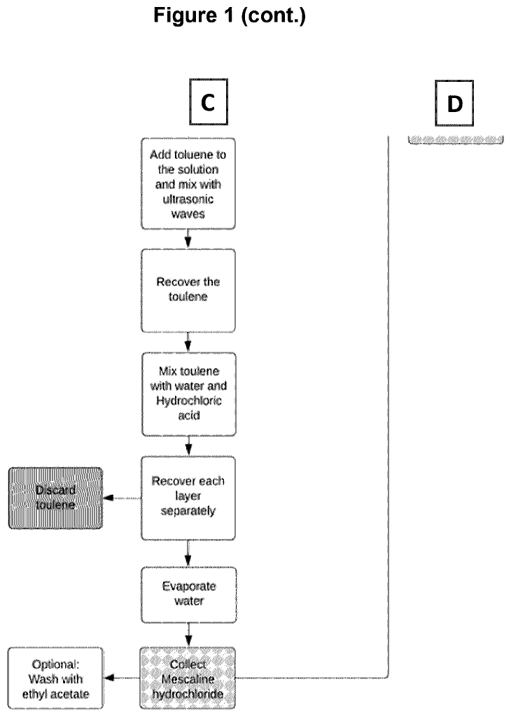

In one embodiment, an extraction process of the disclosure is represented in the flow chart of FIG. 1.

In certain plants, the active compounds can be found throughout the plant. Accordingly, in one embodiment of the disclosure, there is included a process for the extraction of active compounds from plant material using the full plant in the process as described above.

In one embodiment, the process comprises:
a) drying, optionally by freeze drying, the plant material to a moisture level of less than about 10%;
b) reducing the particle size of the dried plant material to obtain a powder having a reduced particle size;
c) mixing the powder with a non-polar solvent to form a mixture having a solid phase and a liquid phase, wherein the solid phase comprises the active compounds and the liquid phase comprises hydrophobic compounds;
d) agitating, optionally by sonication, the mixture at low power to reduce the particle size of the solid phase;
e) separating the solid phase from the liquid phase;
f) extracting the active compounds from the solid phase by mixing the solid phase with an alcohol to form an alcoholic mixture;
g) separating, optionally by centrifuging, the alcoholic mixture to obtain a first alcoholic extract and a depleted solid phase;
h) optionally further separating, optionally by filtering, the alcoholic extract to obtain a filtered alcoholic extract;
i) optionally repeating steps g)-i) on the depleted solid phase to obtain a second filtered alcoholic extract;
j) combining the first and second alcoholic extracts and removing, optionally by evaporating, the alcohol to obtain a crude oil comprising the active compounds;
k) mixing the crude oil with ethyl acetate and acidified water to form a crude mixture;
l) agitating, optionally by sonication, the crude mixture at low power to obtain a water phase and a first ethyl acetate phase;
m) separating the water phase from the ethyl acetate phase;
n) mixing the water phase with a base to obtain a basified water phase having a basic pH, optionally having a pH greater than 12.0;
o) mixing the basified water phase with ethyl acetate and separating therefrom a second ethyl acetate phase; and
p) combining the first and second ethyl acetate phases and removing the ethyl acetate to obtain a purified crude oil comprising the active compounds.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Prophetic Example 1—Extraction of Mescaline from Cacti

### 1.1—Material Preparation

The skin and cuticle of five kilograms of fresh cut cacti which may have a variance in potency between 0.1% to 10% alkaloid content is removed from the inner, white flesh. Spines and glochids are also removed from the skin.

The skin is then placed in freezer safe vessels in the freezer for about 12 hours, and after which, the vessels are removed from the freezer and the skin thawed to ambient temperature. The freeze/thaw process is repeated three times.

The cuticle is then removed from the skin and the samples are cut into less than or equal to 2.5 cm² chunks. Once the skin is cut into chunks, the samples are placed into a freeze dryer to sublimate moisture for about 24-36 hours. The moisture of the samples is analyzed using a moisture analyzer with a desired moisture of about 0%-10%. The samples are placed in the freezer dryer repeatedly until the desired moisture level. After freeze drying, approximately 100 g of chlorenchyma biomass is obtained which is powderized via milling and placed into a 30 μm filter bag. The filter bag is then ready for ultrasonic bath.

### 1.2—Defatting Wash

The filter bag containing the biomass is placed in the ultrasonic bath with about 653 g of petroleum ether. The biomass is sonicated for about 20 minutes at about 90 W, and the petroleum ether is drained and recovered from the biomass. The biomass in the filter bag is then transferred to a centrifuge and also filled with about 653 g of fresh petroleum ether, and the mixture is agitated for about five minutes. The system is drained and then the centrifuge is spun at about 6000 RPM for about 5 minutes to dry the biomass within the filter bag. The petroleum ether is recovered.

### 1.3—Full-Spectrum Extract

With the biomass in the filter bag in the centrifuge, the biomass is flooded with about 792 g of methanol and soaked for about 20 minutes. The biomass is agitated by switching between clockwise rotation and counter-clockwise rotation of the centrifuge. The system is drained and the centrifuge is spun at about 6000 RPM for about five minutes to dry the biomass within the filter bag. If moisture is detected, the biomass is further spun for additional time, and if necessary, 0-2% methanol is added to the biomass. Approximately one liter of micella is obtained. An eight-inch vacuum assisted buchner funnel with a 30 μm qualitative filter paper and 200 g of diatomaceous earth. The micella is then filtered through the buchner funnel and the filtrate loaded into a rotary evaporator. The mantle of the rotary evaporator is set for about 70° C. and the condenser to a maximum temperature of about −10° C. The micella is allowed to reach equilibrium temperature and the vacuum turned on. After separation, about 792 g of methanol is recovered and about 15 g of concentrated botanical extract.

### 1.4—Separation

An acidic water solution in a beaker is prepared containing 217.8 mL of water and 2.2 mL of 3% hydrochloric acid. In a separate beaker, 198 g of ethyl acetate is prepared. The acidic water is slowly added to the crude botanical extract until a paste begins to form, which must be unform (and similar consistency to wet plaster). Once the paste is of uniform consistency, the paste is added to the beaker containing ethyl acetate. An ultrasonic probe is placed into the solution so that the probe is 5 mm into the surface of the solution. The probe is set at about 90 W and the mixture sonicated for about 20 min. After sonication, the material is loaded into a 2 L separatory funnel, and each layer is separated and collected in a clean beaker. The aqueous phase is neutralized using calcium hydroxide and verified with a pH testing strip. The aqueous phase is then mixed with an additional 198 g of ethyl acetate and then separated in the separatory funnel at least two more times. The aqueous phases are loaded into a rotary evaporator in which the mantle of the rotary evaporator is set for about 100° C. and the condenser to a maximum temperature of about 10° C. The aqueous phase is allowed to reach equilibrium temperature and the vacuum turned on. After separation, about 9.9 g of concentrated botanical extract is recovered and about 220 ml of water.

The accumulated and collected ethyl acetate phases will contain mescaline that can be concentrated into mescaline hydrochloride, separated by chromatography or isolated as mescaline citrate.

### 1.5—Concentration

The ethyl acetate is recovered using a rotary evaporator in which the mantle of the rotary evaporator is set for about 80° C. and the condenser to a maximum temperature of about −10° C. The micella is allowed to reach equilibrium temperature and the vacuum turned on. After separation, about 4.9 g of mescaline hydrochloride is recovered and about 792 g of ethyl acetate.

1.6—Chromatography Separation Separation can be accomplished with flash chromatography by diluting the solution into a mobile phase of chloroform and methanol (10:1 chloroform to methanol). A stationary phase of silica dioxide is used, and argon gas at a pressure of =<10 psi is used.
1.6a—Mescaline Citrate Isolation

If the mescaline has been concentrated as above, it is re-diluted in about 198 g of ethyl acetate and the solution mixed with a stir bar so that a slight vortex is visible, no deeper than 1 cm. The nucleation is started by slowly adding about 1 g of citric acid to the ethyl acetate solution until the solution becomes cloudy. The mixture is mixed for at least 10 minutes until the mixture becomes clear and crystalline structures are seen. The solution is sealed and left to sit for about 72 hours.

An eight-inch vacuum assisted buchner funnel with a 20 μm qualitative filter paper and a non-fibrous 1 μm filter paper is prepared. Some quantity of mescaline citrate may adhere to the glass and must be scraped and recovered, and additional volumes of ethyl acetate can be used to help release the crystals from the glass, and this volume of ethyl acetate must be filtered as well. The mescaline citrate appears as a white powder. It can be washed of any residual contamination, if present, with ethyl acetate chilled to −40 C, or colder. Approximately 4.9 g of mescaline citrate and 198 g ethyl acetate is recovered.

1.6b—Isolated Mescaline Hydrochloride

An acidic water solution in a beaker is prepared containing 297.0 ml of water and 3.0 mL of 3% hydrochloric acid. This solution is added to the ultrasonic bath containing the powdered biomass and set to a temperature of 90° C. The probe is set at about 90 W and the mixture sonicated for about 30 min. The acidic water solution is drained and collected and the biomass bag is placed into the centrifuge with 300 mL of acidic water. The centrifuge is warmed to 90° C. and agitated by switching between a clockwise rotation and a counter-clockwise rotation for 30 minutes. The acidic water now containing alkaloids is drained from the centrifuge and the leftover mixture is centrifuged for 5 minutes at 6000 RPMs. Residual acidic water must not be trapped within the biomass. If any moisture is detected, the material is spun for additional time. The water is collected from the centrifuge and added to the acidic water from sonication. The acidic water solution is then reduced in volume to 45 mL using a hot plate with stirring set to a temperature of 100° C.

An eight-inch vacuum assisted buchner funnel with a 30 μm qualitative filter paper and 200 g of diatomaceous earth is prepared. An alkaline water solution of mass 22.5 g is prepared using 22.5 mL of water and 11.25 g of sodium hydroxide. The acidic water is basified until it reaches a pH of 12 by slowly adding in the alkaline water solution using a pipette. The solution is gently mixed until it is homogeneous and then 200 mL of toluene is added. An ultrasonic probe is placed into the solution so that the probe is 5 mm into the surface of the solution. The probe is set at about 90 W and the mixture sonicated for about 20 min.

The solution is then transferred to a separatory funnel and the layers separated into separate beakers. The above steps are repeated four times. All of the aqueous layers are collected and discarded and all of the toluene layers are combined into a beaker. 100 mL of PH neutral water is mixed into the toluene fraction, and then 0.5 mL aliquots of hydrochloric acid are added until the pH of the solution is 5.0. The solution is then stirred for 10 minutes using a magnetic stirrer and transferred to a separatory funnel. The toluene phase is collected and discarded while the aqueous phase is collected in a separate beaker. The aqueous phase is loaded into a rotary evaporator which is set for about 100° C. and the condenser to a maximum temperature of about 10° C. The solution is allowed to reach an equilibrium temperature and then vacuum turned on. After separation, depending on starting material potency, about 2.5 g of mescaline hydrochloride is recovered. The recovered crystals are washed with −40° C. ethyl acetate in a buchner funnel with 30 μm filter paper. The crystals are slowly washed and then allowed to dry.

1.6c—Mescaline Fumarate

Further purification is possible through a process that results in mescaline fumarate. An acidic water solution is prepared with mass 127.5 mL having 125 ml of water and 2.5 mL of hydrochloric acid (3%). The mescaline hydrochloride is mixed with the acidic water using a magnetic stirrer until all of the mescaline dissolves. While mixing, 125 mL of toluene is added, and allowed to mix for 10 minutes and then poured into a separatory funnel.

An alkaline water solution of mass 175 g is prepared from 125 ml of water and 50 g of sodium hydroxide. The alkaline water is poured into the separatory funnel and mixed for 30 minutes, and the pH brought up to 12. The solution is allowed to separate and each fraction is collected. The aqueous layer is discarded and the toluene later collected in a beaker.

A methanol and fumaric acid solution containing 31.68 mL methanol and 0.5 g fumaric acid for each 1 g of mescaline hydrochloride. A methanol fumaric acid solution is prepared with a mass of 80.45 g containing 79.2 g of methanol and 1.25 g of fumaric acid. This solution is slowly added to the toluene fraction with gentle mixing, and the solution becomes cloudy as mescaline fumarate begins to nucleate in solution. The solution is mixed until the solution becomes clear and all of mescaline fumarate crystals have formed. The solution is then filtered using an 8″ vacuum assisted buchner funnel with a 20 μm qualitative filter paper and a non-fibrous 1 μm filter paper. Some quantity of mescaline fumarate may adhere to the glass and is scraped and recovered. The filtered solution can be reused to help release crystals from the glass, and this solution must be filtered an additional time as well. The mescaline fumarate is collected from the filter paper and dried. Approximately 0.1 g to 5.0 g, or about 0.5 g to 4.0 g, of mescaline fumarate is recovered depending on the starting potency of the cacti.

### Example 2—Extraction of Mescaline from Cacti

### Material Preparation

The glochids and spines of the cacti (Echinopsis Sp.) were removed and discarded, then the skin was carefully removed but avoiding removal of the cacti interior flesh. The cacti “skins” (epidermis and chlorenchyma) were loaded into the freeze dryer for 12 hours. The skins were thawed back to room temperature after 12 hours of freeze drying. The freeze drying process was repeated two more times.

After the process of freeze drying and thawing, the skins were cut into 2.5 cm² pieces. The cuticle was then removed from the cacti skin and loaded into the freeze dryer for 24-36 hours. After 24-36 hours the freeze-dried material was broken down using a scalpel to shave the material down to appropriate size to avoid particle contamination. The measured size was approximately 2.5 mm². The moisture content of the broken-down biomass was verified to be <10%.

The broken-down/powdered material was then added to a mesh bag of =<30 μm pore size. The biomass filled mesh bag was then loaded into an ultrasonic bath.

### Material Wash #1 (Defatting)

The ultrasonic bath containing biomass was flooded with 0.1 polarity index petroleum ether with a boiling point (BP) of 35-60° C. The biomass was placed into the ultrasonic bath with an energy of 90 W for 20 minutes. The biomass was removed from the ultrasonic bath and placed into the centrifuge. The petroleum ether was then removed from the ultrasonic bath. The centrifuge was filled with virgin petroleum ether. The biomass was agitated for 5 minutes in the centrifuge containing the petroleum ether before draining the petroleum ether from the centrifuge. A 5-minute spin cycle at 6000 rpm was run on the centrifuge to dry the biomass before discarding the excess petroleum ether form the centrifuge.

### Material Wash #2 (Full Spectrum Extraction)

The centrifuge containing the biomass was flooded with methanol (0.76 polarity index) and allowed to soak for 20 minutes. The methanol was drained from the centrifuge. A 5-minute spin cycle at 6000 rpm was run on the centrifuge to dry the biomass.

The solution yielded from the 5-minute spin cycle in the centrifuge was run through a Buchner funnel with sintered disk and diatomaceous earth/30 μm filter paper to capture any biomass powder that may have entered the solution. The methanol solution was loaded into a rotary evaporator/36° C. water bath with vacuum assistance to recover the methanol. The crude extract was recovered in a beaker.

### Separation

A 0.1-1% acid solution was prepared using 3% hydrochloric acid (HCl). This is ethyl acetate. The pH of the solution was checked to make sure it was between 3-5.

The acidic water was poured into a beaker and ethyl acetate (220 ml/100 g of botanical extract) was added to the acidic water. The crude botanical extract was slowly added to the acidic water containing ethyl acetate to form a paste using a metal spatula. The consistency of the paste was checked to make sure it was uniform. The paste was then added to a beaker containing ethyl acetate. An ultrasonic probe was set to the surface of the mixture and ran at 90 W. (Alternatively, an ultrasonic bath can be used but the beaker must be sealed with parafilm and the water level in the bath must be level to the surface of the mixture in the beaker).

The mixture was added to a separatory funnel and mixed. The ethyl acetate solution was recovered and stored in a beaker. The aqueous phase was added to a separatory funnel and mixed. The aqueous layer was neutralized using calcium hydroxide (CaOH) and the pH was checked. An additional volume of ethyl acetate (220 ml/100 g of biomass) was added to the separatory funnel and mixed. The ethyl acetate was recovered and stored in the same beaker. The extraction with ethyl acetate was repeated two more times, then the aqueous layer was discarded.

The ethyl acetate in the beaker contained all the alkaloids from the cactus.

A rotary funnel was used to remove the ethyl acetate (BP: 77.1°) from the desirables (alkaloids including mescaline) to make a concentrated crude.

### Mescaline Citrate Isolation

The ethyl acetate solution was placed on a magnetic Stirrer. A magnetic bar was placed into the beaker containing the solution and stirred until a medium vortex was formed. Small amounts of citric acid (5 g at a time) were added to the solution until it became cloudy. At the first sign of cloudiness in the solution the addition of citric acid was stopped. Observation of the solution for 10 minutes was done to yield a translucent solution after being cloudy. This was mescaline citrate.

As the mescaline citrate was formed the beaker was removed from the magnetic stirrer and the magnetic bar removed from the beaker with the solution.

The solution was poured into a sealable glass container, sealed, and placed in an area where it was undisturbed for 72 hours. After 72 hours the solution was run through a Buchner funnel that had 1 μm qualitative filter paper with a 20 μm filter on top. Mild agitation/scraping with a spatula was required to remove the crystals from the surface of the glass container. Fresh ethyl acetate was used to rinse the class container and the solution was run through the Buchner funnel. Post filtration the crystals (mescaline citrate) were collected.

### Material Wash #3 (Isolated Mescaline Hydrochloride)

Ethyl acetate (0.1-1%) was prepared using 3% hydrochloric acid. Two liters of Ethyl acetate was added to 100 g of cacti powder and loaded into an ultrasonic bath at 90° C. for 30 minutes. The bag was removed from the ultrasonic bath and loaded into the centrifuge. The water from the ultrasonic bath was drained into a beaker and set aside.

The temperature of the centrifuge was set to 90° C. and flooded with ethyl acetate (acidic water) and agitated for 30 minutes. The water from the centrifuge was drained and collected in a beaker before running the centrifuge for 5 minutes at 6000 rpm. The excess water was collected into a beaker.

All the aliquots of acidic water were combined and ran through a Buchner funnel with sintered disc to capture any biomass that may have entered the solution. Diatomaceous earth was used as a filter aid. The acidic water solution was reduced to 300 ml/100 g of extracted cacti.

A basic water solution was created using 50 g NaOH in 100 ml of water for every 300 ml of acidic water.

The basic water solution was added to the acidic water solution to basify to a pH of ˜12. The mixture was placed into a beaker and 200 ml of toluene was added to the beaker (200 ml toluene/100 g cacti). An ultrasonic probe was set at the surface of the mixture and ran at 90 W for 20 minutes. (In the absence of an ultrasonic probe the ultrasonic bath could be used by bringing the water level in the bath equivalent to the surface of the mixture in the beaker). The solution was poured into a separatory funnel after 20 minutes with the ultrasonic probe or bath. The toluene was recovered, and the extraction was repeated four times.

100 ml of water was added to the toluene extract (100 ml water/100 g cacti). 0.5 ml of concentrated HCl was added to the solution and properly mixed for 5 minutes. The pH of the mixture was checked to make sure the pH was reduced to 5. (If the pH is still basic (9+) after adding the first 0.5 ml of HCl, additional HCl in 0.5 ml increments can be added until a pH of 5 is achieved). The solution was then added to the separatory funnel and mixed for 10 minutes then the toluene fraction was collected and discarded. Toluene was on the surface of the aqueous layer. The aqueous fraction was collected in a beaker (a round bottom flask is also suitable). The water was evaporated from the flask by placing it into a hot water bath such that direct heat to the flask was avoided. Brownish crystals remained in the flask; they were recovered as these were the mescaline crystals. The crystals were washed with ethyl acetate to remove the brownish color. The ethyl acetate was decanted after the wash. The crystals were filtered through a Buchner funnel with 20 μm filter paper. This was done by chilling the ethyl acetate to −40° C. in a wash bottle, the crystals were placed into the Buchner funnel and slowly washed with the chilled ethyl acetate.

### Purification

A solution containing 50 ml water and 1 ml HCL was prepared (50 ml water:1 ml HCL). 51 ml of this solution was prepared for each 1 g of mescaline HCL. A beaker containing 50 ml water: 1 ml HCL was placed on a magnetic stirrer and a magnetic bar was placed into the beaker flowed by 1 g of mescaline HCL crystals and stirred until the crystals dissolved completely. 50 ml of toluene was added to the beaker for each 1 g of mescaline HCL and mixed well. The solution was then added to the separatory funnel.

A mixture containing 50 ml water and 10 g NaOH and a 5:1 solution for each 1 g of mescaline HCL was prepared. The NaOH and water solution was poured into the separatory funnel and mixed well for 30 minutes. Each layer was separated from the separatory funnel and collected in its own beaker. The toluene layer was saved, and the aqueous layer was discarded.

0.5 g of fumaric acid was added to 40 ml of methanol and mixed well until fumaric acid was completely dissolved. 80 ml of the above solution was added for each 1 g of mescaline HCl. The fumaric methanol solution was added to the toluene layer in a beaker and gently mixed until it became cloudy (mescaline fumarate begin to nucleate). After all the crystals were formed the solution was gently mixed and poured into a Buchner funnel with 30 μm filter paper. The methanol and toluene mixture flowed through the filter paper leaving behind the purified mescaline fumarate crystals. Excess mescaline fumarate crystals that were left behind in the beaker were collected.

Precipitates were dried, weighed and appropriately dissolved to identify specific species of mescaline concentration. All species were weighed gravimetrically prior to being run on our HPLC.

Table 1 (Plant A) shows a comparison between the mass of cacti needed for the extraction of a given amount of mescaline citrate, hydrochloride, and fumarate, using the procedure in Example 2.

Table 2 (Plant B) shows a comparison between the mass of cacti needed for the extraction of a given amount of mescaline citrate, hydrochloride, and fumarate using the procedure in Example 2.

Table 3 (Plant C) shows a comparison between the mass of cacti needed for the extraction of a given amount of mescaline citrate, hydrochloride, and fumarate using the procedure in Example 2.

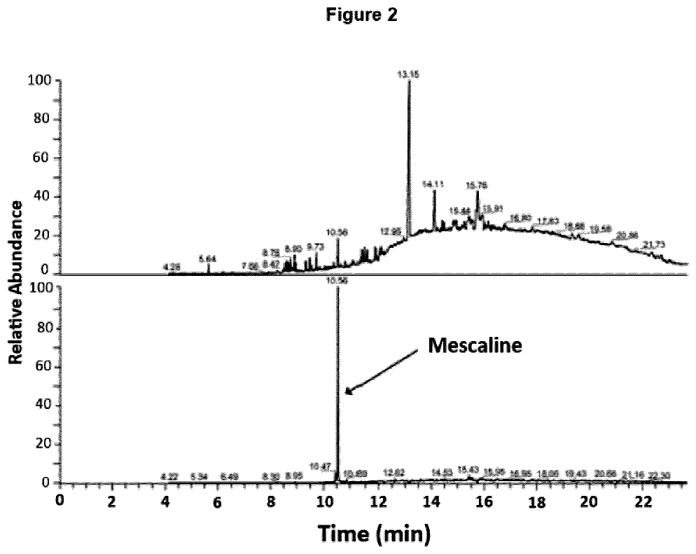

FIG. 2 shows a chromatogram of the mescaline isolated from Plant A using the procedure in Example 2 on a Shimadzu LC-2030 NT (top chart) against a standard of mescaline (from Cayman Chemical). All mescaline species were collected as dry crystallite masses from all plants (A, B and C), dissolved in methanol and subsequently analyzed by HPLC. Top graph is a chromatogram from a sample prepared using the process in Example 2 (Plant A). Chromatograms were obtained for all plants and showed the isolation of mescaline. The bottom Chromatogram represents the standard used from Cayman Chemical.

While the present disclosure has been described with reference to what are presently considered to be the preferred examples, it is to be understood that the application is not limited to the examples described herein. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1
(Plant A) The comparison between the mass of cacti needed
for the extraction of a given amount of mescaline citrate,
hydrochloride, and fumarate. (Horizontal Cut)

| | Mass of | Mass of | Total Mass |
|---|---|---|---|
| | Chemical | Cacti used | of Plant A |
| Chemical | Compound | (g) wet | (g) wet |
| Compounds | Extracted (g) | weight | weight |
| Mescaline Citrate | 4.93 g | 163.6 g | 354.7 g |
| Mescaline | 4.90 g | 163.6 g | 354.7 g |
| Hydrochloride | | | |
| Mescaline Fumarate | 4.91 g | 163.6 g | 354.7 g |

TABLE 2
(Plant B) The comparison between the mass of cacti needed
for the extraction of a given amount of mescaline citrate,
hydrochloride, and fumarate. (Horizontal Cut)

| | Mass of | Mass of | Total Mass |
|---|---|---|---|
| | Chemical | Cacti used | of Plant B |
| Chemical | Compound | (g) wet | (g) wet |
| Compounds | Extracted (g) | weight | weight |
| Mescaline Citrate | 29.70 g | 518.1 g | 1056.6 g |
| Mescaline | 18.22 g | 518.1 g | 1056.6 g |
| Hydrochloride | | | |
| Mescaline Fumarate | 24.10 g | 518.1 g | 1056.6 g |

TABLE 3
(Plant C) The comparison between the mass of cacti needed
for the extraction of a given amount of mescaline citrate,
hydrochloride, and fumarate. (Star Shape Cut)

| | Mass of | Mass of | Total Mass |
|---|---|---|---|
| | Chemical | Cacti used | of Plant C |
| Chemical | Compound | (g) wet | (g) wet |
| Compounds | Extracted (g) | weight | weight |
| Mescaline Citrate | 59.10 g | 1031.5 g | 1569.2 g |
| Mescaline | 36.29 g | 1031.5 g | 1569.2 g |
| Hydrochloride | | | |
| Mescaline Fumarate | 37.94 g | 1031.5 g | 1569.2 g |

1. A process for the extraction of active compounds from plant material, the process comprising:
a) separating or isolating the chlorenchyma from the plant material;
b) drying, optionally by drying, the chlorenchyma to a moisture level of less than 10%;
c) reducing the particle size of the dried chlorenchyma to obtain a powder having a reduced particle size;
d) mixing the powder with a non-polar solvent to form a mixture having a solid phase and a liquid phase, wherein the solid phase comprises the active compounds and the liquid phase comprises hydrophobic compounds;
e) agitating, optionally by sonication, the mixture at low power to reduce the particle size of the solid phase;
f) separating the solid phase from the liquid phase;
g) extracting the active compounds from the solid phase by mixing the solid phase with an alcohol to form an alcoholic mixture;
h) separating, optionally by centrifuging, the alcoholic mixture to obtain a first alcoholic extract and a depleted solid phase;
i) optionally further separating, optionally by filtering, the alcoholic extract to obtain a filtered alcoholic extract;
j) optionally, repeating steps g)-i) on the depleted solid phase to obtain a second filtered alcoholic extract;
k) combining the first and second alcoholic extracts and removing, optionally by evaporating, the alcohol to obtain a crude oil comprising the active compounds;
l) mixing the crude oil with ethyl acetate and acidified water to form a crude mixture;
m) agitating, optionally by sonication, the crude mixture at low power to obtain a water phase and a first ethyl acetate phase;
n) separating the water phase from the ethyl acetate phase;
o) mixing the water phase with a base to obtain a basified water phase having a basic pH, optionally having a pH greater than 12.0;
p) mixing the basified water phase with ethyl acetate and separating therefrom a second ethyl acetate phase; and
q) combining the first and second ethyl acetate phases and removing the ethyl acetate to obtain a purified crude oil comprising the active compounds.

2. The process according to claim 1, wherein the purified crude oil is further mixed with an acid to crystallize the active compounds.

3. The process according to claim 2, wherein the crystallized active compounds are filtered.

4. The process according to claim 1, wherein the plant material is a cannabis plant, or an Echinopsis plant or fungi.

5. The process according to claim 1, wherein the active compounds are cannabinoids.

6. The process according to claim 1, wherein the active compounds are mescaline and/or other alkaloids.

7. The process according to claim 1, wherein the chlorenchyma is freeze dried to a moisture level of between 0-10.0%.

8. The process according to claim 1, wherein the chlorenchyma are reduced in particle size by milling, pulverizing or grinding.

9. The process according to claim 1, wherein the mixture in step (e) is sonicated at a power of 50 watts to 100 watts.

10. The process according to claim 9, wherein the mixture is sonicated at a power of 90 watts.

11. The process according to claim 1, wherein the solid phase is separated from the liquid phase in step (f) by centrifugation.

12. The process according to claim 1, wherein the alcohol in step (g) is methanol.

13. The process according to claim 1, wherein the alcoholic extract in step (i) is filtered through diatomaceous earth.

14. The process according to claim 1, wherein the crude mixture in step (m) is sonicated at a power of 50 watts to 100 watts.

15. The process according to claim 14, wherein the crude mixture is sonicated at a power of 90 watts.

16. The process according to claim 1, wherein the non-polar solvent is petroleum ether, dichloromethane, pentane, hexane or mixtures thereof.

17. The process according to claim 16, wherein the non-polar solvent is petroleum ether.
